(19) United States
(12) Patent Application Publication
(10) Pub. No.: US 2002/0164360 A9
(43) Pub. Date: Nov. 7, 2002

(54) Use of polyamino acid derivatives to treat seborrhoea and the associated skin disorders

(76) Inventors: Michel Philippe, Wissous (FR); Sylvie Cupferman, L'Hay Les Roses (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/770,473

(22) Filed: Jan. 29, 2001

(30) Foreign Application Priority Data
Jan. 28, 2000 (FR) 00 01210

(51) Int. Cl. A61K 6/00; A61K 7/00; A61K 7/06; A61K 7/11; A61K 31/74

(52) U.S. Cl. 424/401; 424/70.1; 424/70.8; 424/70.17; 424/78.03; 424/78.05; 424/78.06; 424/78.07

(57) ABSTRACT

Processes for treatment of at least one condition chosen from seborrhoea of the skin and scalp, disorders associated with seborrhoea, and disorders associated with microorganisms of the genus Propionibacterium comprising applying to an area in need of said treatment at least one compound chosen from certain polyamino acid derivatives. Processes for the manufacture of a composition for treatment of at least one condition chosen from seborrhoea of the skin and scalp, disorders associated with seborrhoea, and disorders associated with microorganisms of the genus Propionibacterium, said process comprising including in said composition at least one certain polyamino acid derivative. Anti-seborrhoeic and anti-acne compositions comprising at least one certain polyamino acid derivative.

The present invention relates to cosmetic and pharmaceutical compositions such as, for example, dermatological compositions, comprising certain polyamino acid derivatives, as well as a process for treating seborrhoea and the skin disorders associated therewith through the use of these compositions.

The secretion of sebum is a normal and useful phenomenon of the skin and the scalp. However, the hypersecretion of sebum, which is known as seborrhoea, results in unpleasant effects and occasionally a skin pathology, such as a greasy and even acneic skin, and a seborrhoeic condition of the scalp. Sebaceous hypersecretion and the disruption of keratinization of the pilosebaceous follicles may result in obstruction of the pilosebaceous follicles and the formation of retentive lesions and comedones.

These skin disorders, such as acne and hyperseborrhoea, are especially involved in the colonization of the skin and the hair follicles by microorganisms of the genus Propionibacterium such as Propionibacterium acnes, Propionibacterium granulosum and Propionibacterium avidum.

To combat these pathogenic agents, active agents such as triclosan, hexamidine, hexetidine and benzalkonium chloride are commonly used. However, the use of these active agents is not without side effects. For example, triclosan has appreciable toxicity, even when used topically. In addition, it has been found to be insufficiently effective, especially in certain vehicles in which its activity is inhibited by surfactants. Hexamidine and hexetidine in the form of salts are sensitizing substances which may cause allergies. Moreover, benzalkonium chloride may be found to be irritant at the doses at which it is usually used. Furthermore, it destabilizes compositions containing anionic surfactants.

It is thus found that there is still a need for topical active agents which may have an effect on the pathologies associated with the microorganisms of the genus Propionibacterium, and which have an action that may be at least as effective as the compounds of the prior art, while at the same time not having at least some of the drawbacks of the known compounds. One aim of the present invention is therefore to propose specific compounds which may obtain this effect.

One subject of the invention is thus the use of at least one polyamino acid derivative of formula (I) as defined below, and the salts thereof, for the cosmetic treatment of seborrhoea of the skin and scalp, and of skin disorders associated with seborrhoea, and of disorders associated with the microorganisms of the genus Propionibacterium, such as Propionibacterium acnes and Propionibacterium granulosum.

Another subject of the invention is the use of at least one polyamino acid derivative of formula (I) as defined below, and the salts thereof, for the manufacture of a composition, such as a pharmaceutical composition, intended for treating seborrhoea of the skin and scalp, and the skin disorders associated with seborrhoea, and disorders associated with the microorganisms of the genus Propionibacterium, such as Propionibacterium acnes and Propionibacterium granulosum.

A further subject of the invention is a process for treatment, such as a cosmetic treatment, of seborrhoea and skin disorders associated therewith, in which a composition comprising at least one polyamino acid derivative of formula (I) as defined below is applied to the skin and the scalp.

Yet aAnother subject of the invention is a composition comprising, in a physiologically acceptable medium, an effective amount of at least one polyamino acid derivative of formula (I) as defined below, as anti-seborrhoeic active agents and as anti-acne active agents.

The skin disorders associated with seborrhoea may be, for example, seborrhoeic dermatitis, acne, greasy skin with a tendency towards acne and hyperseborrhoea.

It has in fact been found that the polyamino acid derivatives according to the invention may have strong activity on, for example, Propionibacterium acnes and Propionibacterium granulosum, and can thus be used in cosmetic and pharmaceutical compositions, such as dermatological compositions, for example as an anti-seborrhoeic and anti-acne active agent.

Another advantage of the polyamino acid derivatives used in the present invention can be their clearly defined and characterized chemical structure, as a result of which the reproducibility of their manufacture may be easy and their industrial feasibility may also be relatively simple. Furthermore, they may have good solubility and compatibility with the media commonly used in cosmetics, such as, for example, aqueous media.

The polyamino acid derivatives used in the context of the present invention are well known in the prior art, especially in the cosmetics field, such as, for example, for their moisturizing properties, and for their use in haircare. Mention may thus be made of Japanese patent application JP-07/041 467, which discloses a class of polyamino acids of high molecular weight consisting essentially of cysteine, as well as the process for preparing these polyamino acids. A class of polyamino acids characterized by the presence of thiol and disulphide functions has also been disclosed in Japanese patent application JP-06/248 072. These polyamino acids react with the thiol linkages of keratin, thus forming disulphide bridges, which makes it possible to increase the sheen and coloration qualities of the hair. Polyamino acids consisting essentially of amino acids with neutral and acidic chains have been disclosed in Japanese patent application JP-04/198 114, along with their use as moisturizing agents.

Mention may also be made, for example, of patent application FR 2 776 510, which discloses a cosmetic composition intended for reinforcing and caring for keratin fibres, such as the hair, comprising polyamino acid derivatives.

The polyamino acid derivatives correspond to formula (I) below:

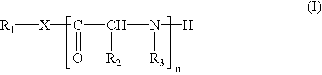

in which:

X is chosen from O, S, NH and NR″ wherein R″ is chosen from saturated and unsaturated, linear and branched C₁₋₆, hydrocarbon-based radicals;

R₁ is chosen from,

(i) hydrogen;

(ii) linear and branched, saturated and unsaturated C₁₋₄₀ hydrocarbon-based radicals, optionally substituted with at least one hydroxyl radical and at least one radical —NRR′ and optionally interrupted with at least one hetero atom chosen from N, O and Si, wherein R and R′, which may be identical or different, are chosen from hydrogen and saturated and unsaturated, linear and branched C₁₋₆ hydrocarbon-based radicals;

(iii) radicals of the formula

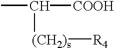

wherein s may be equal to 0, 1, 2, 3 and 4; and R₄ is chosen from hydrogen and radicals chosen from —NH₂, —OH, —SH, —CHOHCH₃, —CONH₂, —NH—C(NH₂)═NH, —C₆H₅, —C₆H₄OH and

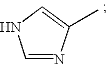

and

(iv) radicals of the formula

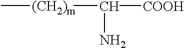

wherein m may be equal to 3, 4 and 5;

R₂ is chosen from hydrogen; saturated and unsaturated, linear and branched C₁₋₈ hydrocarbon-based radicals; radicals chosen from —CH₂C₆H₅, —CH₂C₆H₄OH, —CH₂OH, CHOHCH₃, —(CH₂)ₜ—NH₂ wherein t may be equal to 3, 4 and 5;

R₃ is chosen form hydrogen and saturated and unsaturated, linear and branched C₁₋₆ hydrocarbon-based radicals;

n is a number greater than 1 such that the number average molecular weight of the polyamino acid derivative generally ranges from 100 to 200 000;

wherein the repeating units may be identical or different for the same derivative.

For example, the repeating units may be identical. If the repeating units are different, then at least one of R₂ and R₃ may be varied between the repeating units by choosing at least one of the other meanings given for these radicals.

The salts of the polyamino acid derivatives, such as mineral acid salts and organic acid salts, also form part of the present invention.

According to one embodiment of the present invention, at least one of the following definitions apply to the polyamino acid derivatives:

X is chosen from O, S, NH and NR″, wherein R″ is chosen from saturated and unsaturated, linear and branched C₆ hydrocarbon-based radicals;

R₁ is chosen from linear and branched, saturated and unsaturated C₈₋₄₀ hydrocarbon-based radicals, optionally substituted with at least one hydroxyl radical and one radical —NRR′ and optionally interrupted with at least one hetero atom chosen from N, O and Si, wherein R and R′, which may be identical or different, may be chosen from hydrogen and saturated and unsaturated, linear and branched C₁₆ hydrocarbon-based radicals;

R₂ is hydrogen;

R₃ is chosen from saturated, linear and branched C₁₋₆ hydrocarbon-based radicals; such as, for example, methyl and ethyl radicals;

n is chosen from a number ranging from 2 to 100, or is chosen from a number such that the number average molecular weight of the polyamino acid derivative generally ranges from 150 to 10 000. In certain embodiments, each of these definitions apply.

In another embodiment of the present invention, at least one of the following definitions apply to the polyamino acid derivatives:

X is chosen from O, S and NH;

R₁ is chosen from linear and branched, saturated C₁₀₋₂₄ hydrocarbon-based radicals, optionally substituted with 1, 2, 3 and 4 hydroxyl radicals; and linear and branched C₁₂₋₂₄ hydrocarbon-based radicals comprising at least one double unsaturation, optionally substituted with at least one hydroxyl radical;

R₂ is hydrogen;

R₃ is a methyl radical; and

n is chosen from a number ranging from 4 to 50, or is chosen from a number such that the number average molecular weight of the polyamino acid derivative generally ranges from 300 to 8 000. In certain embodiments, each of these definitions apply.

The polyamino acid derivatives according to the invention may readily be prepared by those skilled in the art on the basis of their general knowledge. Patent application FR 2 776 510, for example, discloses a process for preparing these compounds.

The polyamino acid derivatives may be present in the composition, such as a cosmetic and pharmaceutical composition, in an amount which is sufficient to obtain the desired effect, such as in an amount generally ranging from 0.001% to 30% by weight, for example, such as from 0.01 % and 15% by weight, relative to the total weight of the composition. In one embodiment, the polyamino acid derivatives are present in the composition in an amount ranging from 0.5% to 5% by weight, relative to the total weight of the composition.

The compositions comprising the polyamino acid derivatives moreover comprise a physiologically acceptable medium, in particularly cosmetically and pharmaceutically acceptable mediums, i.e., a medium which is compatible with the skin, mucous membranes, the hair and the scalp.

They may be in any presentation form which is suitable for topical application, such as in the form of aqueous, aqueous-alcoholic, organic and oily solutions; aqueous, aqueous-alcoholic and oily gels; pasty and solid anhydrous products; water-in-oil, oil-in-water and multiple emulsions; suspensions and dispersions in solvents and fatty substances, of lotion and serum type; microemulsions, microcapsules, microparticles; vesicular dispersions of ionic type (liposomes) and of nonionic type; and mousses.

The physiologically acceptable medium in which the polyamino acid derivatives may be used, and its constituents, their amount, the presentation form of the composition and its mode of preparation, may be chosen by those skilled in the art on the basis of their general knowledge depending on the desired type of composition and the desired use.

For example, the composition may comprise any fatty substance usually used in the field of application envisaged. Mention may be made of silicone fatty substances such as silicone oils, gums and waxes, as well as non-silicone fatty substances such as oils and waxes of plant, mineral, animal and synthetic origin. The oils may optionally be volatile and non-volatile. Mention may also be made of hydrocarbons, synthetic esters and ethers, fatty alcohols and fatty acids.

The composition can also comprise an aqueous medium, an aqueous-alcoholic medium containing an alcohol such as ethanol and isopropanol, an organic medium comprising common organic solvents such as C₁₋₆ alcohols, for example ethanol and isopropanol, glycols such as propylene glycol, and ketones.

The composition can further comprise at least one conventional emulsifier chosen from amphoteric, anionic, cationic and nonionic emulsifiers, used alone and as a mixture.

The composition can also comprise at least one adjuvant that is common in the field under consideration, such as hydrophilic and lipophilic thickeners and gelling agents, hydrophilic and lipophilic additives, active agents, such as cosmetic active agents, preserving agents, antioxidants, fragrances, fillers, pigments, UV screening agents, odor absorbers, dyes, moisturizers (glycerol), vitamins, essential fatty acids, liposoluble polymers, such as hydrocarbon-based liposoluble polymers, opacifiers, stabilizers, sequestering agents, conditioners and propellants.

Needless to say, a person skilled in the art will take care to select the optional at least one adjuvant and the amount thereof such that the advantageous properties of the composition according to the invention are not substantially adversely affected by the addition envisaged.

For example, when the composition of the invention is an emulsion, the proportion of the fatty phase can generally range from 5% to 80% by weight, such as from 5% to 50% by weight, relative to the total weight of the composition. The oils, emulsifiers and co-emulsifiers used in the composition in emulsion form are chosen from those used conventionally in cosmetics and dermatology. The emulsifier and optionally the co-emulsifier may be present in the composition in a proportion generally ranging from 0.3% to 30% by weight, such as from 0.5% to 20% by weight, relative to the total weight of the composition. The emulsion can also contain lipid vesicles.

Among the oils which may be used, mention may be made of mineral oils (liquid petroleum jelly), plant oils (liquid fraction of karite butter), animal oils, synthetic oils (purcellin oil, hydrogenated polyisobutene), silicone oils and fluoro oils (perfluoropolyethers).

Among the emulsifiers which may be used, mention may be made of fatty acid esters of polyols, such as fatty esters of sorbitol, for instance sorbitan tristearate sold under the name Span 65 by the company ICI, and fatty esters of glycerol such as glyceryl monostearate, and the glycol palmitostearate/polyethylene glycol stearate (6 EO)/polyethylene glycol stearate (32 EO) mixture sold under the name “Tefose 63” by the company Gattefosse; hydrogenated lecithin; polyethylene glycol (PEG) esters such as PEG-40 stearate sold under the name Myrj 52 by the company ICI. They may also be silicone emulsifiers such as the cetyldimethicone copolyol sold under the name Abil EM90 by the company Goldschmidt.

Among the hydrophilic gelling agents which may be mentioned are natural gums (xanthan gum), polysaccharides (hydroxypropylmethylcellulose, carboxymethylcellulose), carboxyvinyl polymers (carbomer), acrylic copolymers such as acrylate/alkylacrylate copolymers, polyglyceryl (meth)acrylates such as the product sold under the name Norgel by the company Guardian, polyacrylamides and the mixture of polyacrylamide, C₁₃₋₁₄-Isoparaffin and Laureth-7, sold under the name Sepigel 305 by the company SEPPIC, oxyethylenated sugar derivatives such as oxyethylenated methylglucose; lipophilic gelling agents which may be mentioned are modified clays such as bentones, metal salts of fatty acids, hydrophobic silica and polyethylenes.

Among the hydrophilic and lipophilic active agents which may be used in the above compositions, mention may be made of active agents which may complement the effect of the polyamino acid derivatives in the treatment of seborrhoea and of associated disorders, such as acne.

These may be, for example, antiinflammatory agents such as benzoyl peroxide; antibiotics such as clindamycin and erythromycin; antiseptic agents such as octopirox; keratolytic active agents such as salicylic acid and its derivatives, α-hydroxy acids, β-hydroxy acids, retinoic acid and its derivatives, retinol and its derivatives; antiseborrhoeic agents such as di- and trivalent metal salts, for instance alkaline-earth metal salts and lanthanide salts.

Moreover, hydrophilic active agents which may be used, for example, are proteins and protein hydrolysates, amino acids, polyols (glycerol, propylene glycol), urea, allantoin, sugars and sugar derivatives, water-soluble vitamins, starch, bacterial and plant extracts, and moisturizers. Lipophilic active agents which may be used, for example, are tocopherol and its derivatives, essential fatty acids, sphingolipids and essential oils.

When skin comprising disorders associated with seborrhoea has to be exposed to sunlight, the composition comprising the polyamino acid derivatives under consideration may also comprise at least one sunscreen, so as to preserve the efficacy of the derivative according to the invention while at the same time protecting the skin against the harmful effects of the sun's rays. Among the sunscreens which may be used, mention may be made of pigments which may optionally be in the form of nanoparticles (nanopigments), and in particular metal oxides such as titanium oxide, iron oxide and zinc oxide. Among the organic screening agents which may be mentioned are sulphonic and sulphonate derivatives of benzophenone, sulphonic and sulphonated derivatives of benzylidenecamphor and acrylates such as octocrylene. The amount of screening agent depends on the desired sun protection and can generally range from 0.01% to 10% by weight, such as from 0.1% to 5% by weight, relative to the total weight of the composition.

The pH of the compositions according to the invention may generally be less than 7, such as from 3 to 6.

The derivatives according to the invention find an application in, for example, compositions which may be:

in the form of dermatological and cosmetic skincare products for the face, the body including the scalp, and the lips, such as care base for the lips, antisun protective compositions and artificial tanning compositions; care and treatment compositions (day products, night products, anti-wrinkle products, moisturizers, etc.) for the face; matt-effect compositions for the face; cleansing and make-up-removing gels and creams; protective body milk and bodycare milk; purifying milk and lotions; and cover sticks;

in the form of make-up products for the skin of the face, the body and the lips, such as foundations, concealer sticks and cover sticks;

in the form of aftershave gels and lotions;

in the form of pharmaceutical compositions;

in the form of solid compositions such as cleansing soaps and cleansing bars;

in the form of haircare compositions, such as medicated shampoos and medicated lotions, such as, for example, those which are anti-seborrhoeic.

The polyamino acid derivatives according to the invention find application in, for example, compositions intended for treating acne, greasy skin, and greasy scalps, and thus in so-called anti-acne and anti-seborrhoeic compositions.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The invention is illustrated in greater detail in the nonlimiting examples which follow.

### EXAMPLE 1

The compound of formula (I) was prepared in which R₁=C₈H₁₇—CH═CH—C₈H₁₆—, X=—NH—, R₂=H, R₃=—CH₃and n=9.8.

46 g (0.4 mol) of sarcosine

N-carboxyanhydride was suspended in 500 ml of toluene in a 1-liter reactor. 13 g (0.05 mol) of oleylamine was then added dropwise. At the end of the addition, the mixture was maintained at 80° C. for about 2 hours. It was then cooled to room temperature, after which 50 ml of ethanol (95° C.) were added.

After evaporation of the solvents under reduced pressure and drying under vacuum, 42 g of a powder were obtained.

The index “n” was determined by NMR.

By varying the proportion of oleylamine, the polyamino acid derivative having the same structure but with an index “n” equal to 41 was also prepared.

### EXAMPLE 2

The antimicrobial activity of the two compounds prepared in Example 1 was determined with respect to Propionibacterium acnes and Propionibacterium granulosum.

The steps followed in carrying out this test were as follows:

1) culturing the microorganism: Propionibacterium acnes and Propionibacterium granulosum were cultured on slanted tryptocasein soya agar;

2) preparation of the inoculum: 4 days before the start of the test, the two bacterial strains were subcultured on a suitable medium and were left to incubate for 4 days at 35° C. under anaerobic conditions. On the day of the test, the slant was washed with about 9 ml of diluent. The suspension obtained had a titre of 10⁸ microorganisms/ml.

3) preparation of the sample: on the day before the test, 32 g of tryptocasein soya broth were placed in a glass flask known as a pill bottle and were incubated at 35° C. On the day of the test, 4 g of the test compound and 4 ml of inoculum (i.e. 10⁷ microorganisms/ml) were added; homogenization was carried out and the pill bottle was placed in an agitated incubator at 35° C. In parallel, a control was prepared to check that the microorganisms were under favourable growth conditions throughout the test.

4) sampling and counting: after 24 hours of contact, the contents of the pill bottle were homogenized and 1 g was taken therefrom. After determining the appropriate dilution to be able to carry out the counting, this dilution was spread onto the surface of agar Petri dishes (Eugon LTIOO medium) and the Petri dishes were then left to incubate for 24 hours in an incubator at 35° C. under anaerobic conditions.

The colonies were then counted on the dishes containing more than 20 and less than 200 colonies.

The test composition (pH 7) comprised the following constituents:

| | |
|---|---|
| carboxyvinyl polymer | 0.3 g |
| sterile distilled water | 98.4 g |
| triethanolamine | 0.3 g |
| test compound | 1 g |

The results obtained were indicated in the table which follows. They were expressed as the number of microorganisms that were revivable after 24 hours, per gram of preparation:

| | Propionibacterium | Propionibacterium |
|---|---|---|
| Composition | acnes | granulosum |
| Composition A | 1.1 × 10⁶ | < 20 |
| (compound of Ex. 1, | | (sensitivity threshold of |
| n = 9.8) | | the method) |
| Composition B | 1.8 × 10⁵ | 2.2 × 10³ |
| (compound of Ex. 1, | | |
| n = 41) | | |
| Control | 4.4 × 10⁷ | 1.3 × 10⁸ |

It was thus found that the compounds according to the invention clearly showed significant activity with respect to Propionibacterium acnes and Propionibacterium granulosum.

This result was moreover confirmed by a second test which showed that after 24 hours of contact, composition A had reduced the initial Propionibacterium granulosum population by 5.8 log₁₀, and composition B by 4.8 log₁₀ compared with the placebo.

### EXAMPLE 3

### Foaming Gel for Seborrhoeic Greasy Skin

| | |
|---|---|
| compound of Example 1 (n = 9.8) | 1% |
| copolymer of oxyethylenated (60 EO) hydrogenated tallow | 0.9% |
| alcohol/myristyl glycol (solubilizing agent) (Elfacos | |
| GT 282 S from Akzo) | |
| glycerol | 3% |
| glycolic acid at 57% by weight in water | 0.5% |
| N-disodium N-carboxyethoxyethyl | 5% |
| N-cocoylamidoethyl aminoacetate at 38% in water | |
| sodium lauryl ether sulphate at 28% in water | 14.3% |
| sodium chloride | 1% |
| coconut fatty acid diethanolamide (softener) | 0.7% |
| fragrance | qs |
| oxyethylenated (26 EO) oxypropylenated (26 PO) butyl | 1% |
| alcohol, oxyethylenated (40 EO) hydrogenated castor oil | |
| mixture | |
| demineralized water | qs 100% |

The gel obtained was suitable for treating seborrhoeic dermatitis, by application twice a day to the face.

### EXAMPLE 4

### Foaming Cleansing Cream for Acneic Skin

| | |
|---|---|
| ethylene glycol monostearate | 2% |
| compound of Example 1 (n = 41) | 0.5% |
| magnesium aluminium silicate hydrate | 1.7% |
| hydroxypropylmethylcellulose | 0.8% |
| mixture of sodium cocoyl isethionate and of coconut fatty | 15% |
| acids (Elfan AT 84 G from Akzo) | |
| stearic acid | 1.2 5% |
| sodium lauroyl sarcosinate at 30% in water | 10% |
| fragrance | qs |
| demineralized water | qs 100% |

The cream thus obtained was suitable for cleansing acneic skin, for example by use on the face twice a day.

### EXAMPLE 5

### Medicated Cream for Acne

| | |
|---|---|
| sorbitan tristearate | 1% |
| compound of Example 1 (n = 9.8) | 1.5% |
| crosslinked carboxyvinyl homopolymer | 0.4% |
| xanthan gum | 0.5% |
| ethylene glycol dimethacrylate/lauryl methacrylate | 1% |
| copolymer | |
| cyclopentadimethylsiloxane | 6% |
| glycerol | 3% |
| emulsifier | 4% |
| fragrance | qs |
| demineralized water | qs 100% |

The cream obtained was suitable for treating the skin, by application to the face and the back once a day.

### EXAMPLE 6

### Medicated Gel for Seborrhoeic Skin

| | |
|---|---|
| compound of Example 1 (n = 41) | 1% |
| xanthan gum | 1% |
| glycerol | 2% |
| ethanol | 20% |
| oxyethylenated (26 EO) oxypropylenated (26 PO) butyl | 1% |
| alcohol, oxyethylenated (40 EO) hydrogenated castor oil | |
| mixture in water | |
| fragrance | qs |
| demineralized water | qs 100% |

This gel was suitable for treating seborrhoeic skin, by application to the face once or twice a day.

### EXAMPLE 7

### Tinted Medicated Cream for Acneic Skin

| | |
|---|---|
| hydrogenated lecithin | 2.4% |
| apricot kernel oil | 6% |
| ethylene glycol dimethacrylate/lauryl methacrylate | 1% |
| copolymer | |
| oxyethylenated (5 EO) soybean sterols | 1.6% |
| compound of Example 1 (n = 41) | 1% |
| iron oxides | 0.9% |
| titanium oxide | 5% |
| polyacrylamide/C₁₃-C₁₄-Isoparaffin/Laureth-7 | 4% |
| (Sepigel 305) | |
| cyclopentadimethylsiloxane | 6% |
| glycerol | 6% |
| propylene glycol | 6% |
| fragrance | qs |
| demineralized water | qs 100% |

This cream was beige-coloured and was suitable for treating the skin, by application to the face twice a day.

### EXAMPLE 8

### Cover Stick for Greasy Skin

| | |
|---|---|
| waxes (carnauba wax and ozokerite) | 14% |
| liquid fraction of karite butter | 4% |
| titanium oxide and zinc oxide | 22% |
| iron oxides | 4% |
| compound of Example 1 (n = 9.8) | 1% |
| polydimethylsiloxane/hydrated silica | 0.1% |
| cetyl alcohol | 1.4% |
| isoparaffin | qs 100% |

The stick obtained can be applied to the face several times a day.

What is claimed is:

1 . A process for treatment of at least one condition chosen from seborrhoea of the skin and scalp, disorders associated with seborrhoea, and disorders associated with microorganisms of the genus Propionibacterium, said process comprising:
applying to an area in need of said treatment at least one compound chosen from polyamino acid derivatives of formula (I) and salts thereof, 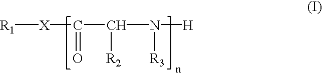
in which:
X is chosen from O, S, NH and NR″ wherein R″ is chosen from saturated and unsaturated, linear and branched C₁₋₆ hydrocarbon-based radicals;
R₁ is chosen from:
(i) hydrogen;
(ii) linear and branched, saturated and unsaturated C₁₋₄₀ hydrocarbon-based radicals,
(iii) radicals of the formula 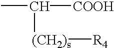
wherein s is a number chosen from 0, 1, 2, 3 and 4; and R₄ is chosen from hydrogen and radicals chosen from —NH₂, —OH, —SH, —CHOHCH₃, —CONH₂, —NH—C(NH₂)═NH, —C₆H₅, —C₆H₄OH and 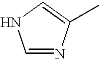
and;
(iv) radicals of the formula 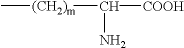
wherein m is a number chosen from 3, 4 and 5;
R₂ is chosen from hydrogen; saturated and unsaturated, linear and branched C₁₋₈ hydrocarbon-based radicals; and radicals chosen from —CH₂C₆H₅, —CH₂C₆H₄OH, —CH₂OH, —CHOHCH₃, —(CH₂)ₜ—NH₂, wherein t is a number chosen from 3, 4 and 5;
R₃ is chosen from hydrogen and saturated and unsaturated, linear and branched C₁₋₆ hydrocarbon-based radicals; and
n is a number greater than 1 chosen such that the number average molecular weight of the polyamino acid derivative ranges from 100 to 200 000;
wherein the repeating unit may be identical or different for the same derivative.

2. A process according to claim 1, wherein said microorganisms are Propionibacterium acnes.

3. A process according to claim 1, wherein said microorganisms are Propionibacterium granulosum.

4. A process according to claim 1, wherein R₁ is chosen from linear and branched, saturated and unsaturated C₁₋₄₀ hydrocarbon-based radicals substituted with at least one hydroxyl radical, at least one radical —NRR′, or at least one hydroxyl radical and at least one radical —NRR′, wherein R and R′, which may be identical or different, are chosen from hydrogen and saturated and unsaturated, linear and branched C₁₋₆ hydrocarbon-based radicals.

5. A process according to claim 1, wherein R₁ is chosen from linear and branched, saturated and unsaturated C₁₋₄₀ hydrocarbon-based radicals interrupted with at least one hetero atom chosen from N, O and Si.

6. A process according to claim 1, wherein said at least one compound is administered in the form of a cosmetic composition.

7. A process according to claim 6, wherein the treatment comprises the cosmetic treatment of at least one disorder chosen from seborrhoeic dermatitis, acne, greasy skin with a tendency towards acne, and hyperseborrhoea.

8. A process according to claim 1, wherein said at least one compound is administered in the form of a pharmaceutical composition.

9. A process according to claim 8, in which the pharmaceutical composition is administered for treating at least one disorder chosen from seborrhoeic dermatitis, acne, greasy skin with a tendency towards acne and hyperseborrhoea.

10. A process according to claim 1, wherein in said polyamino acid derivatives of formula (I) and salts thereof, at least one of the following definitions apply:
X is chosen from O, S, NH and NR″, wherein R″ is chosen from saturated and unsaturated, linear and branched C₁₋₆ hydrocarbon-based radicals;
R₁ is chosen from linear and branched, saturated and unsaturated C₈₋₄₀ hydrocarbon-based radicals,
R₂ is hydrogen;
R₃ is chosen from saturated, linear and branched C₁₋₆ hydrocarbon-based radicals; and
n is chosen from a number ranging from 2 to 100 and a number chosen such that the number average molecular weight of said polyamino acid derivative ranges from 150 to 10,000.

11. A process according to claim 10, wherein R₃ is chosen from methyl and ethyl radicals.

12. A process according to claim 10, wherein R₁ is chosen from linear and branched, saturated and unsaturated C₈₋₄₀ hydrocarbon-based radicals substituted with at least one hydroxyl radical, at least one radical —NRR′, or at least one hydroxyl radical and at least one radical —NRR′, wherein R and R′, which may be identical or different, are chosen from hydrogen and saturated and unsaturated, linear and branched C₁₋₆ hydrocarbon-based radicals.

13. A process according to claim 10, wherein R₁ is chosen from linear and branched, saturated and unsaturated C₈₋₄₀ hydrocarbon-based radicals interrupted with at least one hetero atom chosen from N, O and Si.

14. A process according to claim 14, wherein n is chosen from a number ranging from 2 to 100.

15. A process according to claim 14, wherein n is a number chosen such that the number average molecular weight of said polyamino acid derivative ranges from 150 to 10,000.

16. A process according to claim 10, wherein:
X is chosen from O, S, NH and NR″, wherein R″ is chosen from saturated and unsaturated, linear and branched C₁₋₆ hydrocarbon-based radicals;
R₁ is chosen from linear and branched, saturated and unsaturated C₈₋₄₀ hydrocarbon-based radicals,
R₂ is hydrogen;
R₃ is chosen from saturated, linear and branched C₁₋₆ hydrocarbon-based radicals; and
n is chosen from a number ranging from 2 to 100 and a number chosen such that the number average molecular weight of said polyamino acid derivative ranges from 150 to 10,000.

17. A process according to claim 1, wherein in said polyamino acid derivatives of formula (I) and salts thereof, at least one of the following definitions apply:
X is chosen from O, S and NH;
R₁ is chosen from linear and branched, saturated C₁₀₋₂₄ hydrocarbon-based radicals; and linear and branched unsaturated hydrocarbon-based radicals;
R₂ is hydrogen;
R₃ is a methyl radical; and
n is chosen from a number ranging from 4 to 50 and a number chosen such that the number average molecular weight of said polyamino acid derivative ranges from 300 to 8,000.

18. A process according to claim 17, wherein n is chosen from a number ranging from 4 to 50.

19. A process according to claim 17, wherein n is a number chosen such that the number average molecular weight of said polyamino acid derivative ranges from 300 to 8,000.

20. A process according to claim 17, wherein X is NH.

21. A process according to claim 17, wherein R₁ is chosen from linear and branched, saturated C₁₀₋₂₄ hydrocarbon-based radicals substituted with at least one hydroxyl radical.

22. A process according to claim 21, wherein said linear and branched, saturated C₁₀₋₂₄ hydrocarbon-based radicals are substituted with 1, 2, 3, or 4 hydroxyl radicals.

23. A process according to claim 17, wherein R₁ is chosen from linear and branched unsaturated hydrocarbon-based radicals substituted with at least one hydroxyl radical.

24. A process according to claim 1, wherein:
X is chosen from O, S and NH;
R₁ is chosen from linear and branched, saturated C₁₀₋₂₄ hydrocarbon-based radicals; and linear and branched unsaturated hydrocarbon-based radicals;
R₂ is hydrogen;
R₃ is a methyl radical; and
n is chosen from a number ranging from 4 to 50 and a number chosen such that the number average molecular weight of said polyamino acid derivative ranges from 300 to 8,000.

25. A process according to claim 1, wherein said at least one compound is present in said composition in an amount ranging from 0.001 % to 30% by weight, relative to the total weight of the composition.

26. A process according to claim 25, wherein said at least one compound is present in said composition in an amount ranging from 0.01 % to 15% by weight, relative to the total weight of the composition.

27. A process according to claim 26, wherein said at least one compound is present in said composition in an amount ranging from 0.5% to 5% by weight, relative to the total weight of the composition.

28. A process according to claim 10, wherein said at least one compound is applied in the form of a composition chosen from a cosmetic composition and a pharmaceutical composition.

29. A process according to claim 17, wherein said at least one compound is applied in the form of a composition chosen from a cosmetic composition and a pharmaceutical composition.

30. A process according to claim 1,wherein said at least one compound is applied to at least one area chosen from the skin and the scalp.

31. A process for the manufacture of a composition for treatment of at least one condition chosen from seborrhoea of the skin and scalp, disorders associated with seborrhoea, and disorders associated with microorganisms of the genus Propionibacterium, said process comprising:
including in said composition at least one polyamino acid derivative chosen from formula (I) and salts thereof, 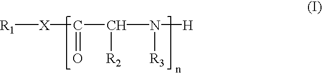
in which:
X is chosen from O, S, NH and NR″ with R″ is chosen from saturated and unsaturated, linear and branched C₁₋₆ hydrocarbon-based radicals;
R₁ is chosen from:
(i) hydrogen;
(ii) linear and branched, saturated and unsaturated C₁₋₆ hydrocarbon-based radicals,
(iii) radicals of the formula 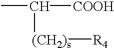
wherein s is a number chosen from 0, 1, 2, 3 and 4; and R₄ is chosen from hydrogen and radicals chosen from —NH₂, —OH, —SH, —CHOHCH₃, —CONH₂, —NH—C(NH₂)═NH, —C₆H₅, —C₆H₄OH and 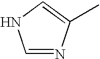
and;
(iv) radicals of the formula 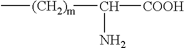
wherein m is a number chosen from 3, 4 and 5;
R₂ is chosen from hydrogen; saturated and unsaturated, linear and branched C₁₋₈ hydrocarbon-based radicals; and radicals chosen from —CH₂C₆H₅, —CH₂C₆H₄OH, —CH₂OH, —CHOHCH₃, —(CH₂)ₜ—NH₂ wherein t is a number chosen from 3, 4 and 5;
R₃ is chosen from hydrogen and saturated and unsaturated, linear and branched C₁₋₆ hydrocarbon-based radicals; and
n is a number greater than 1 chosen such that the number average molecular weight of the polyamino acid derivative ranges from 100 to 200 000;
wherein the repeating unit may be identical or different for the same derivative.

32. A process according to claim 31, wherein said microorganisms are Propionibacterium acnes.

33. A process according to claim 31, wherein said microorganisms are Propionibacterium granulosum.

34. A process according to claim 31, wherein R₁ is chosen from linear and branched, saturated and unsaturated C₁₋₄₀ hydrocarbon-based radicals substituted with at least one hydroxyl radical, at least one radical —NRR′, or at least one hydroxyl radical and at least one radical —NRR′, wherein R and R′, which may be identical or different, are chosen from hydrogen and saturated and unsaturated, linear and branched C₁₋₆ hydrocarbon-based radicals.

35. A process according to claim 31, wherein R₁ is chosen from linear and branched, saturated and unsaturated C₁₋₄₀ hydrocarbon-based radicals interrupted with at least one hetero atom chosen from N, O and Si.

36. A process according to claim 31, wherein in said polyamino acid derivatives of formula (I) and salts thereof, at least one of the following definitions apply:
X is chosen from O, S, NH and NR″, wherein R″ is chosen from saturated and unsaturated, linear and branched C₁₋₆ hydrocarbon-based radicals;
R₁ is chosen from linear and branched, saturated and unsaturated C₈₋₄₀ hydrocarbon-based radicals,
R₂ is hydrogen;
R₃ is chosen from saturated, linear and branched C₁₋₆ hydrocarbon-based radicals; and
n is chosen from a number ranging from 2 to 100 and a number chosen such that the number average molecular weight of said polyamino acid derivative ranges from 150 to 10,000.

37. A process according to claim 36, wherein R₃ is chosen from methyl and ethyl radicals.

38. A process according to claim 36, wherein R₁ is chosen from linear and branched, saturated and unsaturated C₈₋₄₀ hydrocarbon-based radicals substituted with at least one hydroxyl radical, at least one radical —NRR′, or at least one hydroxyl radical and at least one radical —NRR′, wherein R and R′, which may be identical or different, are chosen from hydrogen and saturated and unsaturated, linear and branched C₁₋₆ hydrocarbon-based radicals.

39. A process according to claim 36, wherein R₁ is chosen from linear and branched, saturated and unsaturated C₈₋₄₀ hydrocarbon-based radicals interrupted with at least one hetero atom chosen from N, O and Si.

40. A process according to claim 36, wherein n is chosen from a number ranging from 2 to 100.

41. A process according to claim 36, wherein n is a number chosen such that the number average molecular weight of said polyamino acid derivative ranges from 150 to 10,000.

42. A process according to claim 36, wherein:
X is chosen from O, S, NH and NR″, wherein R″ is chosen from saturated and unsaturated, linear and branched C₁₋₆ hydrocarbon-based radicals;
R₁ is chosen from linear and branched, saturated and unsaturated C₈₋₄₀ hydrocarbon-based radicals,
R₂ is hydrogen;
R₃ is chosen from saturated, linear and branched C₁₋₆ hydrocarbon-based radicals; and
n is chosen from a number ranging from 2 to 100 and a number chosen such that the number average molecular weight of said polyamino acid derivative ranges from 150 to 10,000.

43. A process according to claim 31, wherein in said polyamino acid derivatives of formula (I) and salts thereof, at least one of the following definitions apply:
X is chosen from O, S and NH;
R₁ is chosen from linear and branched, saturated C₁₀₋₂₄ hydrocarbon-based radicals; and linear and branched unsaturated hydrocarbon-based radicals;
R₂ is hydrogen;
R₃ is a methyl radical; and
n is chosen from a number ranging from 4 to 50 and a number chosen such that the number average molecular weight of said polyamino acid derivative ranges from 300 to 8,000.

44. A process according to claim 43, wherein n is chosen from a number ranging from 4 to 50.

45. A process according to claim 43, wherein n is a number chosen such that the number average molecular weight of said polyamino acid derivative ranges from 300 to 8,000.

46. A process according to claim 43, wherein X is NH.

47. A process according to claim 43, wherein R₁ is chosen from linear and branched, saturated C₁₀₋₂₄ hydrocarbon-based radicals substituted with at least one hydroxyl radical.

48. A process according to claim 47, wherein said linear and branched, saturated C₁₋₂₄ hydrocarbon-based radicals are substituted with 1, 2, 3, or 4 hydroxyl radicals.

49. A process according to claim 43, wherein R₁ is chosen from linear and branched unsaturated hydrocarbon-based radicals substituted with at least one hydroxyl radical.

50. A process according to claim 31, wherein said at least one polyamino acid derivative is present in said composition in an amount ranging from 0.001% to 30% by weight, relative to the total weight of the composition.

51. A process according to claim 50, wherein said at least one polyamino acid derivative is present in said composition in an amount ranging from 0.01% to 15% by weight, relative to the total weight of the composition.

52. A process according to claim 51, wherein said at least one polyamino acid derivative is present in said composition in an amount ranging from 0.5% to 5% by weight, relative to the total weight of the composition.

53. A process according to claim 31, wherein said composition is a pharmaceutical composition.

54. An anti-seborrhoeic composition comprising,
a physiologically acceptable medium; and
an effective amount of at least one polyamino acid derivative of formula (I) and salts thereof, 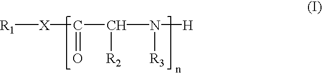
in which:
X is chosen from O, S, NH and NR″ wherein R″ is chosen from saturated and unsaturated, linear and branched C₁₋₆ hydrocarbon-based radicals;
R₁ is chosen from:
(i) hydrogen;
(ii) linear and branched, saturated and unsaturated C₁₋₄₀ hydrocarbon-based radicals,
(iii) radicals of the formula 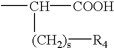
wherein s is a number chosen from 0, 1, 2, 3 and 4; and R₄ is chosen from hydrogen and radicals chosen from —NH₂, —OH, —SH, —CHOHCH₃, —CONH₂, —NH—C(NH₂)═NH, —C₆H₅, —C₆H₄OH and 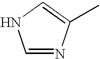
and;
(iv) radicals of the formula 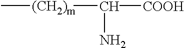
wherein m is a number chosen from 3, 4 and 5;
R₂ is chosen from hydrogen; saturated and unsaturated, linear and branched C₁₋₈ hydrocarbon-based radicals; and radicals chosen from —CH₂C₆H₅, —CH₂C₆H₄OH, —CH₂OH, —CHOHCH₃, —(CH₂)ₜ—NH₂ wherein t is a number chosen from 3, 4 and 5;
R₃ is chosen from hydrogen and saturated and unsaturated, linear and branched C₁₋₆ hydrocarbon-based radicals; and
n is a number greater than 1 chosen such that the number average molecular weight of the polyamino acid derivative ranges from 100 to 200 000;
wherein the repeating unit may be identical or different for the same derivative.

55. An anti-seborrhoeic composition according to claim 54, wherein said composition is an anti-acne composition.

56. An anti-bacterial composition comprising,
a physiologically acceptable medium; and
an effective amount of at least one polyamino acid derivative of formula (I) and salts thereof for treating bacteria, 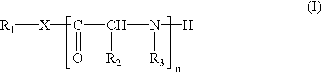
in which:
X is chosen from O, S, NH and NR″ wherein R″ is chosen from saturated and unsaturated, linear and branched C₁₋₆ hydrocarbon-based radicals;
R₁ is chosen from:
(i) hydrogen;
(ii) linear and branched, saturated and unsaturated C₁₋₄₀ hydrocarbon-based radicals,
(iii) radicals of the formula 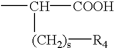
wherein s is a number chosen from 0, 1, 2, 3 and 4; and R₄ is chosen from hydrogen and radicals chosen from —NH₂, —OH, —SH, —CHOHCH₃, —CONH₂, —NH—C(NH₂)═NH, —C₆H₅, —C₆H₄OH and 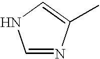
and;
(iv) radicals of the formula 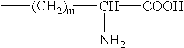
wherein m is a number chosen from 3, 4 and 5;
R₂ is chosen from hydrogen; saturated and unsaturated, linear and branched C0-8 hydrocarbon-based radicals; and radicals chosen from —CH₂C₆H₅, —CH₂C₆H₄OH, —CH₂OH, —CHOHCH₃, —(CH₂)ₜ—NH₂ wherein t is a number chosen from 3, 4 and 5;
R₃ is chosen from hydrogen and saturated and unsaturated, linear and branched C₁₋₆ hydrocarbon-based radicals; and
n is a number greater than 1 chosen such that the number average molecular weight of the polyamino acid derivative ranges from 100 to 200 000;
wherein the repeating unit may be identical or different for the same derivative.

57. An anti-bacterial composition according to claim 56, wherein said composition is an anti-acne composition.

58. An antibacterial composition according to claim 55, wherein the bacteria is of the genus Propionibacterium.

59. An antibacterial composition according to claim 58, wherein the bacteria is at least one of Propionibacterium acnes and Propionibacterium granulosum.
